(19) United States
(12) Patent Application Publication
(10) Pub. No.: US 2014/0364896 A1
(43) Pub. Date: Dec. 11, 2014

(54) DEVICE, SYSTEM, AND METHOD FOR THROMBUS RETRIEVAL

(71) Applicant: Abott Cardiovascular Systems, Inc., Santa Clara, CA (US)

(72) Inventor: Paul M. Consigny, San Jose, CA (US)

(21) Appl. No.: 13/913,280

(22) Filed: Jun. 7, 2013

(51) Int. Cl. A61B 17/3207

(52) U.S. Cl. CPC A61B 17/3207; A61B 2017/00778
USPC 606/200

(57) ABSTRACT

Implementations of the present invention provide devices, systems, and methods for engaging tissue and/or cells forming an occlusion in a body lumen (e.g., in a vessel). More specifically, embodiments of the present invention involve securing at least a portion of the occlusion as well as removing at least a portion of the occlusion from the body lumen. Accordingly, devices, systems, and methods described herein may increase passageway through the body lumen, which, in some instances, may improve and/or at least partially restore fluid flow therethrough.

### CROSS-REFERENCE TO RELATED APPLICATIONS

N/A.

### BACKGROUND OF THE INVENTION

1. The Field of the Invention

This invention relates to systems, methods, and apparatus for retrieving obstructions and/or occlusions, such as a thrombus, from a body lumen.

2. Background and Relevant Art

An obstruction or occlusion, such as a thrombus, present in a body lumen (e.g., in a vessel) may be undesirable, dangerous, and even lethal. For example, a thrombus may obstruct the flow of blood through the vessel. In some instances, reduction of the blood flow may be reduced enough to cause symptoms due to decreased oxygen that may result from the reduced blood flow.

Moreover, complete or substantially complete occlusion of the vessel may result in anoxia or complete deprivation of oxygen. Deprivation of oxygen, in turn, may lead to necrosis or tissue death or infarction. Because occlusions may occur in any number of vessels that may supply blood to various tissues, any number of tissues may be affected by such occlusions. Thus, for instance, occlusions occurring in vessels supplying blood to the heart or brain may be lethal. Consequently, removal or reduction of the occlusions or thrombi from the vessel may be beneficial in some instances and may prevent infarctions. Accordingly, manufacturers and physicians desiring to treat and/or remove occlusions in vessels continue to seek improved methods and apparatuses for such treatment.

### BRIEF SUMMARY OF THE INVENTION

Implementations of the present invention provide devices, systems, and methods for engaging tissue and/or cells forming an occlusion in a body lumen (e.g., in a vessel). More specifically, embodiments of the present invention involve securing at least a portion of the occlusion as well as removing at least a portion of the occlusion from the body lumen. Accordingly, devices, systems, and methods described herein may increase passageway through the body lumen, which, in some instances, may improve and/or at least partially restore fluid flow therethrough.

One embodiment includes an expandable and collapsible thrombus removal device configured to engage and at least partially remove a thrombus. The thrombus removal device may include a selectively expandable capturing portion including a plurality of capturing cells defined by one or more roughened struts. The thrombus removal device also may include a selectively expandable pass-through portion that has a plurality of pass-through cells defined by one or more smooth struts. Furthermore, the pass-through portion may be connected to the capturing portion, and the roughened struts may have a higher surface roughness than the smooth struts. Moreover, each of the plurality of pass-through cells may be larger than each of the plurality of capturing cells. The thrombus removal device may further include a connector portion connected to one or more of the capturing portion or the pass-through portion, and a control wire connected to the connector portion.

In additional or alternative embodiments, the thrombus removal device the density of capturing cells in the capturing portion may be higher than density of pass-through cells in the pass-through portion. Also, the one or more smooth struts may include a coating. For example, the coating may be Poly(vinylidene fluoride-co-hexafluoropropene). Additionally or alternatively, one or more roughened struts may be uncoated. In some embodiments, the one or more roughened struts may have a positive charge. Moreover, the one or more smooth struts may have a negative charge. Embodiments also include the one or more roughened struts having an Arithmetic Mean Roughness (Rₐ), which is a recognized parameter of roughness, of greater than 1 μm. Alternative or additional embodiments may include one or more smooth struts with an Arithmetic Mean Roughness of less than 0.5 μm. Embodiments may further include the capturing portion that may have a tubular shape including an open distal end. In some embodiments, the pass-through cells of the pass-through portion may be in fluid communication with the open distal end of the capturing portion.

One or more embodiments involve a method of removing a thrombus from a body lumen. The method may include inserting a capturing portion of a thrombus removal device into the thrombus, while maintaining at least part of a pass-through portion of the thrombus removal device outside of the thrombus. The pass-through portion may include a plurality of pass-through cells. The method may further include pressing one or more roughened struts of the capturing portion into the thrombus by reconfiguring the capturing portion of the thrombus removal device into a deployed configuration. In addition, the method may include receiving at least a portion of the thrombus inside one or more of a plurality of capturing cells of the capturing portion or an inner space of the capturing portion. Furthermore, the plurality of capturing cells may be defined by the one or more roughened struts, and each of the plurality of the capturing cells may be smaller than each of the plurality of pass-through cells. The method also may include securing at least a portion of the thrombus inside one or more of the plurality of capturing cells of the capturing portion or the inner space of the capturing portion.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention may be obtained, a more particular description of the invention briefly described above will be rendered by reference to specific embodiments thereof which are illustrated in the appended drawings. For better understanding, the like elements have been designated by like reference numbers throughout the various accompanying figures. Understanding that these drawings depict only typical embodiments of the invention and are not therefore to be considered to be limiting of its scope, the invention will be described and explained with additional specificity and detail through the use of the accompanying drawings in which:

FIG. 1A illustrates a perspective view of a cutaway portion of a thrombus removal device in a deployed configuration in accordance with an embodiment of the present invention;

FIG. 1B illustrates a partial side view of a thrombus removal device in a pre-deployed configuration positioned within a catheter tube in accordance with an embodiment of the present invention;

FIG. 2 illustrates a cutaway enlarged side view of roughened struts of a thrombus removal device in accordance with an embodiment of the present invention;

FIG. 3 illustrates a cutaway enlarged side view of smooth struts of a thrombus removal device in accordance with an embodiment of the present invention;

FIG. 4A illustrates a side view of a body lumen and a catheter tube with a thrombus removal device positioned inside the body lumen in accordance with an embodiment of the present invention;

FIG. 4B illustrates a side view of the body lumen of FIG. 4A with the catheter and the thrombus removal device positioned within a thrombus inside the body lumen in accordance with an embodiment of the present invention;

FIG. 4C illustrates a side view of the body lumen of FIG. 4A with the thrombus removal device in a deployed configuration positioned within the thrombus in accordance with an embodiment of the present invention;

FIG. 4D illustrates a side view of the body lumen of FIG. 4A with the thrombus removal device partially retracted into the catheter tube in accordance with an embodiment of the present invention;

FIG. 4E illustrates a side view of the body lumen of FIG. 4A with the thrombus removal device retracted into the catheter tube and securing the thrombus in accordance with an embodiment of the present invention;

FIG. 5 illustrates a chart of acts or steps of a method for removing a thrombus in accordance with an embodiment of the present invention.

### DETAILED DESCRIPTION OF THE PREFERRED EMBODIMENTS

Implementations of the present invention provide devices, systems, and methods for engaging tissue and/or cells forming an occlusion in a body lumen (e.g., in a vessel). More specifically, embodiments of the present invention involve securing at least a portion of the occlusion as well as removing at least a portion of the occlusion from the body lumen. Accordingly, devices, systems, and methods described herein may increase passageway through the body lumen, which, in some instances, may improve and/or at least partially restore fluid flow therethrough.

In some embodiments, an occlusion such as thrombus may be at least partially engaged, secured, and removed by a thrombus removal device. For example, a portion of the thrombus removal device may be positioned inside the thrombus. More specifically, a capturing portion of the thrombus removal device may be at least partially positioned inside the thrombus. Subsequently, the capturing portion of the thrombus removal device may be expanded or enlarged into a deployed configuration. As the capturing portion expands into the deployed configuration, the capturing portion may engage and at least partially secure the thrombus.

Furthermore, in the deployed configuration, the thrombus removal device may enlarge or form an opening through the thrombus. In particular, the capturing portion of the thrombus removal device may be hollow or tubular. In some instances, the capturing portion may protrude past a distal side of the thrombus. Hence, when expanded inside the thrombus (i.e., in the deployed configuration), the capturing portion of the thrombus removal device may increase or form an opening through thrombus.

Additionally, the thrombus removal device may provide a fluid connection or communication between the capturing portion and the body lumen near a proximal side of the thrombus. Thus, the fluid in the body lumen may flow through the thrombus removal device (including the capturing portion) and may be channeled past or through the thrombus. As such, for example, the thrombus removal device may increase and/or restore blood flow in the body lumen.

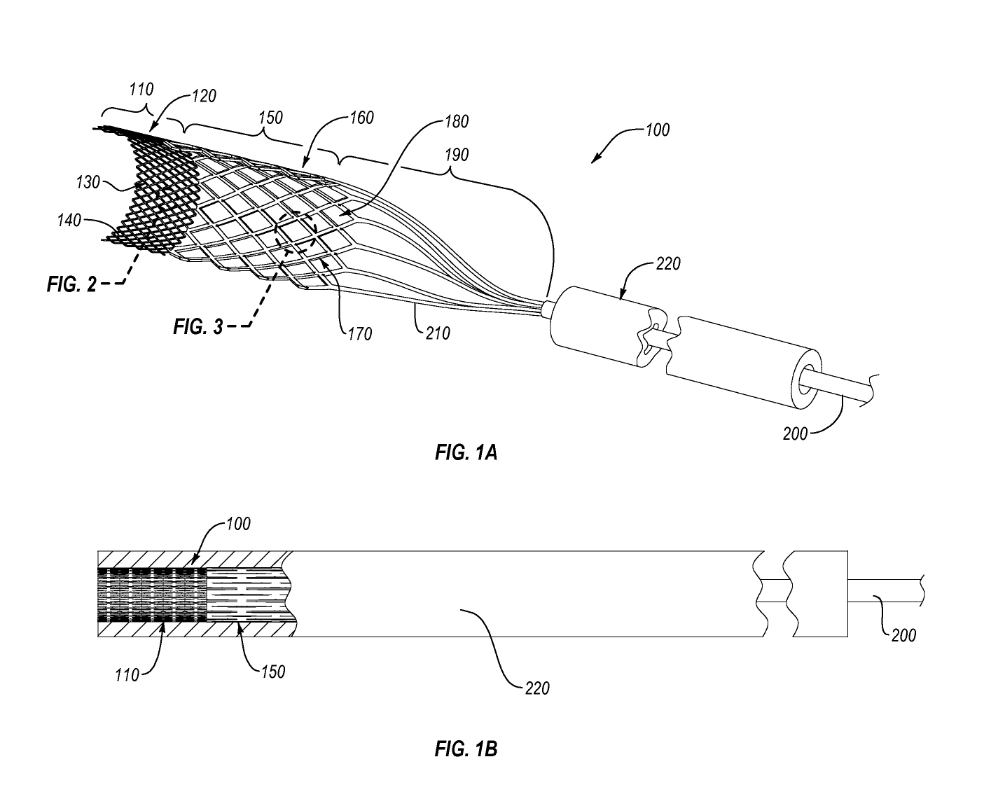

In some embodiments, at least a portion of the thrombus removal device may have a tubular shape. For example, FIG. 1A illustrates an exemplary thrombus removal device 100, which has an approximately tubular shape, in a deployed configuration. In one or more embodiments, as described below in further detail, the thrombus removal device 100 may be expandable into the deployed configuration and collapsible into a pre-deployed configuration. Accordingly, the thrombus removal device 100 may be expanded in a manner that at least a portion of the thrombus removal device 100 engages and secures at least portion of the thrombus, thereby facilitating removal thereof.

In one example, the thrombus removal device 100 may have an approximately cylindrical outer or peripheral surface and/or an approximately cylindrical inner or interior surface, which may define the overall tubular shape thereof. In alternative or additional embodiments, the thrombus removal device 100 or any portion thereof may have number of suitable shapes, such as shapes having elliptical, rectangular, or other cross-sections. Furthermore, a portion of the thrombus removal device 100 may have an approximately cylindrical peripheral surface, and another portion of the thrombus removal device 100 may have a non-cylindrical peripheral surface.

In an embodiment, the thrombus removal device 100 may include a capturing portion 110, which may be configured to engage, secure, and/or capture at least a portion of the thrombus. For example, the capturing portion 110 may have an approximately tubular and/or cylindrical shape. In one or more embodiments, the capturing portion 110 may include a peripheral surface 120 and an interior surface 130, which may have approximately circular cross-sections when the capturing portion 110 and/or the thrombus removal device 100 is in the deployed configuration.

The capturing portion 110 also may include multiple capture cells 140, as described below in further detail. Furthermore, in some embodiments, the capture cells 140 may entirely surround the peripheral surface 120 of the capturing portion 110. Alternatively, the capture cells 140 may be located or positioned only partially about the peripheral surface 120 of the capturing portion 110. For instance, a portion of the peripheral surface 120 may be solid or continuous.

In some embodiments, the capture cells 140 may form or define an opening or a passageway through the peripheral surface 120 and through the interior surface 130 of the capturing portion 110. Accordingly, tissue or cells that form the thrombus may enter and/or pass through the capture cells 140, thereby entering an interior space of the capturing portion 110. In additional or alternative embodiments, at least some of the capture cells 140 may be recessed into the peripheral surface 120 but may not pass through the interior surface 130 of the capturing portion 110. In any event, the capture cells 140 may engage and/or surround the tissue or cells forming the thrombus, thereby securing at least a portion of the thrombus to the capturing portion 110.

As described above, the capturing portion 110 may be reconfigured into the deployed configuration (i.e., expanded) and into the pre-deployed configuration (i.e., contracted). For instance, the capturing portion 110 may be self-expanding, such that the capturing portion 110 may expand into the deployed configuration in the absence of external forces that may restrain or retain the capturing portion 110 in the pre-deployed configuration. Alternatively, the capturing portion 110 may be expanded through application of forces thereon (e.g., with an inflatable balloon, actuating member, etc.).

In the deployed configuration, the peripheral surface 120 of the capturing portion 110 may define a larger surface area than in the pre-deployed configuration. As such, the capture cells 140 also may have or define larger openings or recesses in the peripheral surface 120. In other words, deployment of the capturing portion 110 may result in increase of the openings formed by the capture cells 140. Accordingly, in the deployed configuration, a larger amount of the tissue or cells comprising the thrombus may enter the capture cells 140 then in the pre-deployed configuration.

In some embodiments, the thrombus removal device 100 also may include a pass-through portion 150. The pass-through portion 150 may be connected to the capturing portion 110. In some embodiments, the capturing portion 110 may be located distally relative to the pass-through portion 150. Hence, in an embodiment, the capturing portion 110 may be connected to a distal end of the pass-through portion 150. Alternatively, however, the capturing portion 110 may be located proximally relative to the pass-through portion 150 (e.g., the capturing portion 110 may be connected to a proximal end of the pass-through portion 150). In yet further embodiments, the capturing portion 110 may be surrounded by two pass-through portions 150.

Accordingly, the length of the pass-through portion(s) 150 as well as of the capturing portion 110 may vary from one embodiment to the next. Also, in some embodiments, the pass-through portion 150 may be longer than the capturing portion 110. In alternative or additional embodiments, the pass-through portion 150 may be shorter than the capturing portion 110. For instance, the ratio of the length of the pass-through portion 150 to the length of the capturing portion 110 may be in one or more of the following ranges: between about 10:1 and 5:1; between about 7:1 and 3:1; between about 4:1 and 1:1; between about 2:1 and 1:5; and between about 1:4 and 1:10. In further embodiments, the ratio of the length of the pass-through portion 150 to the length of the capturing portion 110 may be greater than 10:1 or less than 1:10.

In one or more embodiments, at least a portion of the pass-through portion 150 may have approximately the same shape and/or size as at least a portion of the capturing portion 110. Such portions of the pass-through portion 150 and capturing portion 110 may connect together. For example, the pass-through portion 150 may include a peripheral surface 160 and an interior surface 170, at least a portion of which may align with corresponding portions of the peripheral surface 120 and interior surface 130. As such, interior space of the capturing portion 110 (formed or defined by the peripheral surface 120) and interior space of the pass-through portion 150 (formed or defined by the interior surface 170) may be in fluid communication with each other.

Similar to the capturing portion 110, the pass-through portion 150 may be self-expanding and may be capable of expanding into the deployed configuration without application of force thereto. In alternative or additional embodiments, however, the pass-through portion 150 may be expanded through application of force. Furthermore, in some embodiments, the pass-through portion 150 may expand the capturing portion 110. More specifically, as the pass-through portion 150 expands into the deployed configuration, the pass-through portion 150 may force the capturing portion 110 also to expand into the deployed configuration. Alternatively, the capturing portion 110 may force the pass-through portion 150 into the deployed configuration, as the capturing portion 110 expands into the deployed configuration.

Similar to the capturing portion 110, the pass-through portion 150 may include pass-through cells 180. In some instances, the pass-through cells 180 may pass through the peripheral surface 160 and through the interior surface 170. Accordingly, embodiments of the present invention may include the pass-through cells 180 that provide a passageway (e.g., for fluid located in the body lumen) into the interior space of the pass-through portion 150. Therefore, in some embodiments, the thrombus removal device 100 also may include fluid communication between the interior space of the capturing portion 110 and peripheral surface 160 of the pass-through portion 150.

Consequently, in one or more embodiments, the pass-through portion 150 may form a channel across the thrombus so that fluid, such as blood, located on the proximal side of the thrombosis may flow through the thrombus removal device 100 and to the distal side of the thrombus, and vice versa. For example, the fluid may flow into the interior space of the pass-through portion 150 and into the interior space of the capturing portion 110.

In some embodiments, the capturing portion 110 may have an at least partially open distal end 112, such that, for example, fluid located in the interior space of the capturing portion 110 may exit the interior space out of the distal end 112 of the capturing portion 110. Similarly, fluid located near the distal end 112 may pass therethrough and into the interior space of the capturing portion 110. In some instances, the distal end 112 of the capturing portion 110 may be positioned beyond the distal side of the thrombus (i.e., the distal end 112 of the capturing portion 110 may protrude past the thrombus).

As noted above, the pass-through portion 150 may be in fluid communication with the capturing portion 110. Particularly, fluid may pass through the peripheral surface 160 of the pass-through portion 150 and may enter the interior space of the pass-through portion 150 and of the capturing portion 110. Accordingly, the fluid also may pass through and out of the capturing portion 110 (e.g., through the distal end 112 thereof), thereby passing through the thrombus. In other words, the pass-through cells 180 may be in fluid communication with the open distal end 112 of the capturing portion, which may allow the fluid to flow through the pass-through cells 180 and out of the distal end 112 and vice versa. Thus, deployment of the thrombus removal device 100 may increase and/or restore fluid flow through the body lumen, as further described below.

In at least one embodiment, the pass-through cells 180 may be larger than the capture cells 140. For instance, the perimeter of at least some of the capture cells 140 in the deployed configuration may be substantially smaller than the perimeter of at least some of the pass-through cells 180 in the deployed configuration. Hence, pass-through cells 180 may exhibit reduced or minimized binding to the tissue and/or cells surrounding the pass-through portion 150. For instance, blood cells may pass through the pass-through cells 180 and into the interior space of the pass-through portion 150 without binding to the pass-through portion 150.

Additionally or alternatively, the peripheral surface 160 and/or the interior surface 170 of the pass-through portion 150 may be substantially smooth, such as to reduce adhesion of blood cells and/or other tissue or cells thereto. In some embodiments, the pass-through portion 150 may remain in a deployed configuration without or with a minimal accumulation of tissue or cells thereon. As such, the pass-through cells 180 may remain substantially unobstructed to the flow of fluid from the body lumen into the interior space of the pass-through portion 150. In other words, the fluid in the body lumen may continue to flow through the pass-through portion 150 and capturing portion 110 to pass across the thrombus in the body lumen.

In addition, as noted above, the size of the pass-through cells 180 may have substantially greater size than the size of the capture cells 140. For instance, the ratio of the dimensions of the pass-through cells 180 to the dimensions of the capture cells 140 may be in one or more of the following ranges: between about 1.5:1 and 2.5:1; between about 2:1 and 3:1; between about 2.7:1 and 5:1. Embodiments also may include the ratio of the pass-through cells 180 to the capture cells 140 that is less than 1.5:1 and greater than 5:1.

Furthermore, the ratio of capture cells 140 per unit area to the pass-through cells 180 per unit area (i.e., ratio of density of capture cells 140 to the density of pass-through cells 180) may vary from one embodiment to the next. In some embodiments the ratio of capture cells 140 per unit area to the pass-through cells 180 per unit area may be in one or more of the following ranges: between about 1:1 and 2:1; between about 1.5:1 and 3:1; and between about 2.5:1 and 5:1. It should be appreciated, however, that the ratio of capture cells 140 per unit area to the pass-through cells 180 per unit area may be less than 1:1 or greater than 5:1.

In one or more embodiments, the thrombus removal device 100 also may include a connector portion 190, which may connect the pass-through portion 150 and/or to the capturing portion 110 to a control wire 200. The connector portion 190 may include multiple struts or elongated connector members 210 which may connect the pass-through portion 150 and/or the capturing portion 110 to the control wire 200. Additionally or alternatively, the elongated connector members 210 may merge together and/or may be connected or coupled together (e.g., by a sleeve), thereby forming the control wire 200. In other words, the connector portion 190 may be connected to or integrated with the pass-through portion 150 and may be connected to or integrated with the control wire 200.

It should be appreciated that the thrombus removal device 100 may have the capturing portion 110 connected directly to the connector portion 190. In other words, in some embodiments, the thrombus removal device 100 may have no pass-through portion 150. In any event, the connector portion 190 may operatively connect the capturing portion 110 to the control wire 200.

The control wire 200 may allow movement of the thrombus removal device 100 relative to a catheter tube 220. For instance, the control wire 200 may advance the thrombus removal device 100 out of the catheter tube 220 (or may facilitate relative movement of the catheter tube 220 and the thrombus removal device 100) thereby allowing the thrombus removal device 100 to be reconfigured into the deployed or expanded configuration. For instance, at least a portion of the thrombus removal device 100 may include memory shape alloy (e.g., nitinol), which may be in an uncompressed state when the thrombus removal device 100 is in the deployed configuration. Accordingly, advancing the thrombus removal device 100 out of the catheter tube 220 may allow the thrombus removal device 100 to expand into the deployed configuration.

It should be appreciated, however, that the thrombus removal device 100 may be expanded or reconfigured into the deployed configuration in any number of suitable ways. For example, in an unstressed state, the thrombus removal device 100 may be in a pre-deployed configuration. Thus, the thrombus removal device 100 may be forced into the deployed or expanded configuration. In one example, a balloon may be positioned inside the thrombus removal device 100 (e.g., in the interior spaces of the capturing portion 110 and/or pass-through portion 150) when the thrombus removal device 100 is in the pre-deployed configuration. After placing the thrombus removal device 100 at a desired location within the thrombus, the balloon may be inflated inside the thrombus removal device 100 to reconfigure the thrombus removal device 100 into the deployed or expanded configuration.

Additionally, the control wire 200 may allow or facilitate reintroduction of the thrombus removal device 100 into the catheter tube 220. For example, as illustrated in FIG. 1B, after reintroduction of the thrombus removal device 100 into the catheter tube 220, the catheter tube 220 may compress the thrombus removal device 100 into the pre-deployed configuration. Moreover, the thrombus removal device 100 may remain in the pre-deployed configuration within the catheter tube 220. As such, the thrombus removal device 100 may be delivered to the target or the deployment location inside the thrombus together with the catheter tube 220 and may be released from the catheter tube 220 at such location. Subsequently, the thrombus removal device 100 may be reconfigured into the deployed configuration.

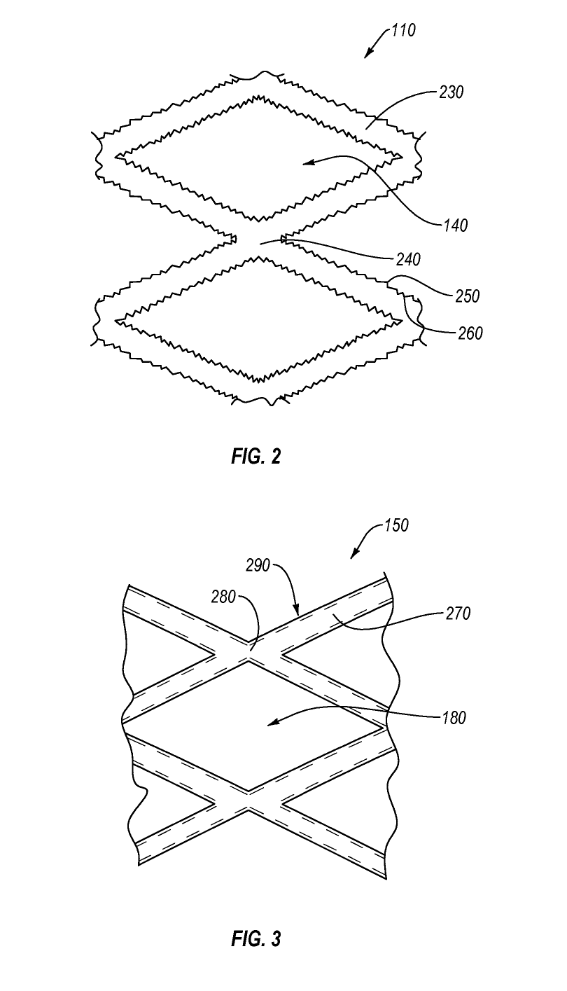

In some embodiments, the capturing portion 110 may include a plurality of struts that may form the wall as well as the peripheral surface 120 and interior surface 130 of the capturing portion 110. Moreover, embodiments also may include struts that have textured or roughened surface. For example, FIG. 2 illustrates an enlarged partial view of the capturing portion 110, which may include interconnected roughened struts 230. More specifically, in an embodiment, the roughened struts 230 may connect to each other at junction points 240. Multiple connections of the roughened struts 230 together may form the capture cells 140.

In an embodiment, roughened struts 230 may include various peaks 250 and/or recesses 260 which may form texture or roughness on the surface of the roughened struts 230. The peaks 250 and recesses 260 may be approximately the same size and/or uniform along the length and/or perimeter of the roughened struts 230. Alternatively, the peaks 250 and/or the recesses 260 may have random and/or varying sizes and shapes. In any event, the peaks 250 and the recesses 260 may form or define roughness on the peripheral surface of the roughened struts 230, which may increase the overall surface area of the roughened struts 230 as compared with smooth struts. Consequently, increased surface area of the roughened struts 230 may increase or improve adhesion of tissue and/or cells of the thrombus, thereby increasing the engagement or connection strength between the capturing portion 110 and the thrombus.

Roughness of the roughened struts 230 may vary from one embodiment to another. For example, the roughened struts 230 may have an Arithmetic Mean Roughness (Rₐ) in one or more of the following ranges: between about 0.8 μm and 2 μm; between about 1 μm and 4 μm; between about 3 and 8 μm; and between about 5 μm and 13 μm. Also, in some instances, the Rₐ of the roughened struts 230 may be greater than 13 μm or less than 0.8 μm. The term “Arithmetic Mean Roughness,” denoted by (Rₐ), refers to an arithmetical mean or average of the heights of minute surface irregularities (i.e., peaks and valleys) from a hypothetical perfect surface or mean line, which has been adopted as the standard measure of surface roughness under ANSI and ASME B46.1-2002.

In one or more embodiments, the height of the peaks 250 may be in one or more ranges of between about 1% and 5%, between about 2% and 15%, between about 10% and 20%, and between about 18% and 30% of the cross-sectional width or length of the roughened struts 230 (e.g., as measured between the recesses 260 across the cross-section of the roughened struts 230). In some embodiments, the height of the peaks 250 may be greater than 30% or less than 1% of the cross-sectional width or length of the roughened struts 230. The peaks 250 and recesses 260 may be formed on the roughened struts 230 in any number of suitable ways. For instance, the roughened struts 230 may be etched (e.g., chemically etched) to form the peaks 250 and recesses 260. In any event, in at least one embodiment, the surface roughness Rₐ of the roughened struts 230 can be sufficient to capture platelets, which can be approximately 2-3 μm in diameter. For instance, the roughened struts 230 can be roughened by pressure blasting the struts with a suitable blasting medium (e.g., sand) to produce a texture or surface roughness desirable and/or suitable for capturing platelets.

In some embodiments, texture or roughened surface may fully surround the roughened struts 230. In other words, the interior and peripheral surfaces of the capturing portion 110 may be roughened. In one or more embodiments, the roughened struts 230 may be partially roughened, such that one or more portions of the roughened struts 230 may be smooth (e.g., the outside or peripheral surface of the roughened struts 230 may be smooth or un-roughened). Moreover, embodiments of the present invention may include roughened struts 230 that have no coating or plating, which may otherwise fill the peaks 250 and recesses 260 and/or which may reduce the overall surface area of the roughened struts 230.

In addition, the capturing portion 110 may include an electrostatic charge, to improve binding of the cells and/or tissue thereto. In an embodiment, one or more of the roughened struts 230 may be electrostatically charged in a manner that attracts the cells (e.g. blood cells) and/or tissue to the roughened struts 230, which may improve or enhance binding of the thrombus to the roughened struts 230 and to the capturing portion 110. For example, the capturing portion 110 and/or at least some of the roughened struts 230 may have a positive electrostatic charge.

The roughened struts 230 may have any number of suitable shapes, widths, and sizes, which may vary from one embodiment to another. In one or more embodiments, the roughened struts 230 may be substantially straight or linear and may have an approximately the same length. Accordingly, connecting four of the roughened struts 230 together may form an equilateral capture cell 140. Furthermore, the roughened struts 230 may have any number of orientations relative to each other. For example, adjacent roughened struts 230 connected at the junction points 240 may be oriented perpendicular to each other. Such configuration may provide approximately square capture cells 140.

It should be appreciated, however, that the particular size and shape of the capture cells 140 may vary from one embodiment to the next. Moreover, any one of the capture cells 140 may be formed by any number of interconnected roughened struts 230. For instance, triangular-shaped capture cells 140 may be formed by three interconnected roughened struts 230.

Additionally, the roughened struts 230 may have non-linear shapes. For example, the roughened struts 230 may have an arcuate shape, a curved shape, a bent shape (e.g., including one or more bends), an irregular shape, and combinations thereof. The particular shape of the capture cells 140, in turn, may vary based on the particular shapes of the roughened struts 230. For example, arcuate roughened struts 230 may form arcuate or circular capture cells 140. Also, a single roughened strut 230 may form one or more of the capture cells 140. For example, the roughened struts 230 may have a looping shape (i.e., including one or more loops), and the loops of the roughened struts 230 may form or define multiple capture cells 140. In any event, the roughened struts 230 may form capture cells 140 of any desirable shapes and/or sizes.

Also, the shapes and/or sizes of the capture cells 140 may vary across the capturing portion 110. In other words, some of the capture cells 140 may have a first shape (e.g., rectangular) and a first size, while other capture cells 140 may have a second shape (e.g., oval) and a second size (the second size may be different from the first size). In any case, however, the roughened struts 230 may form the capture cells 140 that may have suitable shapes and sizes.

Any number of suitable materials and combinations thereof may comprise the capturing portion 110 and the roughened struts 230. Such materials may be superelastic or memory shape alloys, such as Nickel Titanium (or nitinol) alloys. Additionally or alternatively, the roughened struts 230 may be formed from other metallic or non-metallic materials, including but not limited to stainless steel, titanium, plastics, etc.

Furthermore, the junction points 240 of the roughened struts 230 may be formed in various ways, which may vary from one embodiment to another. In one example, the roughened struts 230 may be integrated with one another at the junction points 240. For instance, a solid tubular structure may be perforated (e.g., with a laser, EDM, etc.) to form the roughened struts 230 and the capture cells 140. Alternatively or additionally, wire-shaped roughened struts 230 may be connected together at the junction points 240 (e.g., welded, brazed, twist-connected, etc.) to form the capture cells 140 of the capturing portion 110. For example, a single wire may be wound in a desired pattern and bonded at intersections (or at the junction points 240) within such pattern to form the capture cells 140. In other embodiments, multiple wires may be connected together at the junction points 240 to form the capture cells 140.

Moreover, the roughened struts 230 may have any suitable cross-section, which may vary from one embodiment to the next. For instance, the roughened struts 230 may have an approximately circular cross-section. Alternatively, the roughened struts 230 may have a rectangular, square, or any number of other suitable cross-sections. Furthermore, one or more of the roughened struts 230 may have a first size and shape of the cross-section thereof, while one or more other roughened struts 230 may have a second size and shape of the cross-section thereof, and the first and second size and shape of the cross-sections and may be different one from another.

As described above, the capturing portion 110 may be connected to the pass-through portion 150 (FIGS. 1A-1B). Similar to the capturing portion 110, the pass-through portion 150 may include multiple pass-through cells 180 (FIG. 1A-1B). example, the pass-through portion 150 may include smooth struts 270 interconnected together at junction points 280. Except as otherwise described herein, the smooth struts 270 and the junction points 280 as well as their respective materials, components, or elements may be similar to or the same as the roughened struts 230 and the junction points 240 (FIGS. 1A-2) and their respective materials, components, or elements.

For example, the smooth struts 270 may be substantially smooth (i.e., may have a substantially smooth peripheral or outer surface), such as to reduce adhesion or binding of cells and/or tissue thereto. For instance, the smooth struts 270 may have surface roughness Rₐ in one or more of the following ranges: between about 0.012 μm and about 0.025 μm; between about 0.02 μm and 0.05 μm; between about 0.04 μm and 0.1 μm; between about 0.08 μm and 0.4 μm; between about 0.2 μm and 0.5 μm. In some embodiments, the surface roughness Ra of the smooth struts 270 may be less than 0.012 lam or greater than 0.8 μm. Moreover, embodiments may include smooth struts 270 that have varying roughness, such that a first portion of the smooth strut 270 has a first roughness, while a second portion of the smooth strut 270 has a second, different roughness.

In some embodiments, the surface of the smooth struts 270 may be coated with a coating 290 to cover, conceal, and/or smooth out any surface defects or irregularities as well as to reduce surface roughness. Additionally or alternatively, the coating 290 may neutralize or block surface charges, thereby reducing adhesion or bonding of cells and/or tissue to the surface of the smooth struts 270. Moreover, in one example, the coating 290 may be charged in a manner to repel blood cells (e.g., the coating 290 may have a negative charge). Thus, coating 290 on the smooth struts 270 may provide a substantially smooth surface on and/or around the smooth struts 270.

In one example, such coating 290 may include a polymer coating 290, such as Poly(vinylidene fluoride-co-hexafluoropropene) (“PVDF-HFP”) or similar coating 290. In some embodiments, the coating 290 may be hydrophobic and may have low surface energy. Thus, the coating 290 on the smooth struts 270 may repel fluids, including blood. In any case, the coating 290 on the smooth struts 270 may aid in ensuring that the pass-through cells 180 of the pass-through portion 150 remain substantially unobstructed during the deployment of the thrombus removal device. Furthermore, at least a portion of the smooth struts 270 may include anti-thrombotic coating, such as heparin. In some instance, the smooth struts 270 may have multiple layers of coating, which may reduce surface roughness of the smooth struts 270 as well as provide anti-thrombotic properties thereto.

As noted above, the smooth struts 270 and their materials, components, or elements may be the same as or similar to roughened struts 230 (FIG. 2) and their respective materials, components, or elements, except as described herein. For example, the smooth struts 270 may have approximately the same size and/or shape as the roughened struts 230 (FIG. 2). Alternatively, in some embodiments, the smooth struts 270 may be larger than the roughened struts. For instance, embodiments that include higher density of the capturing cells 140 (FIGS. 1A-2) than the pass-through cells 180 also may include smooth struts 270 that are thicker (or have a larger cross-section) than the roughened struts.

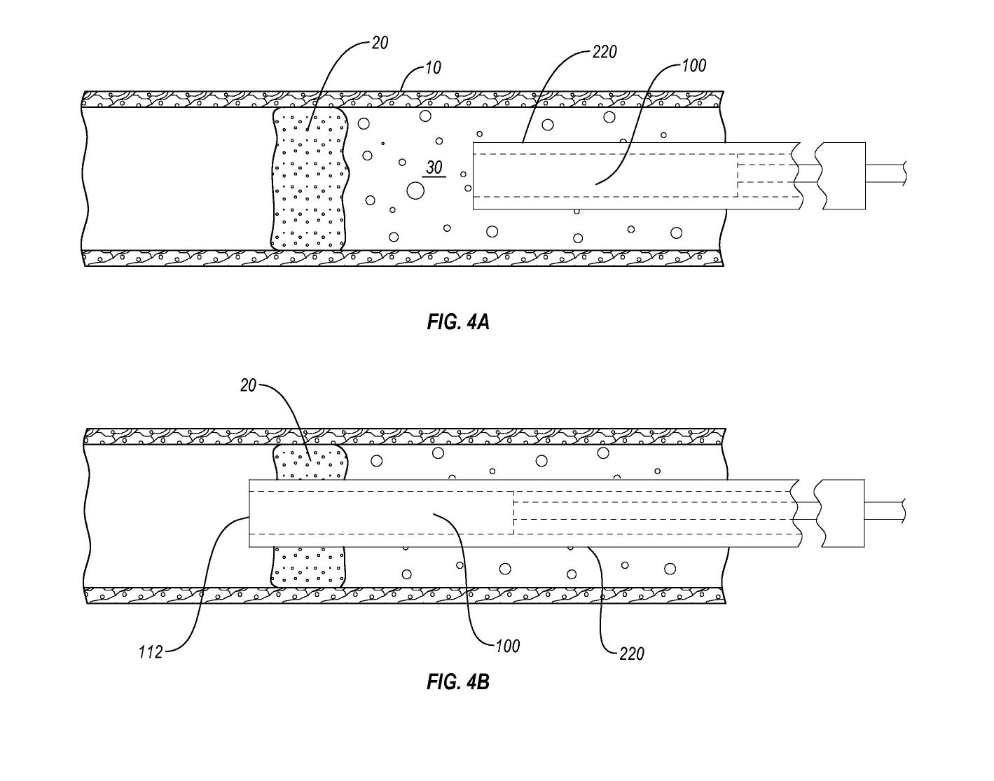

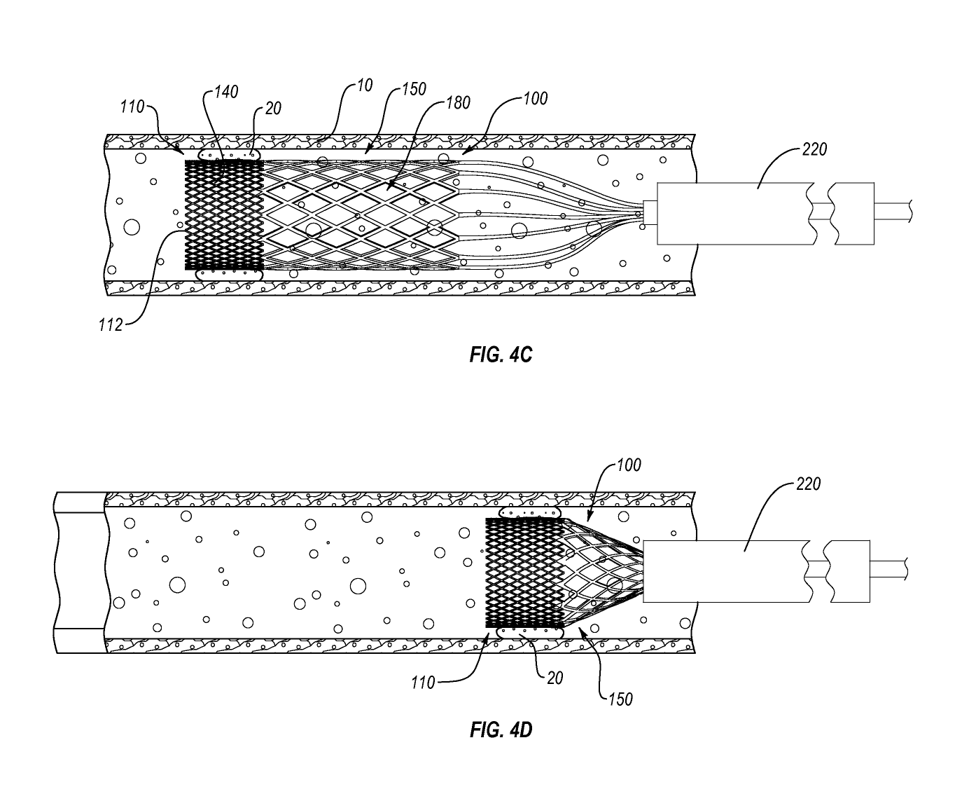

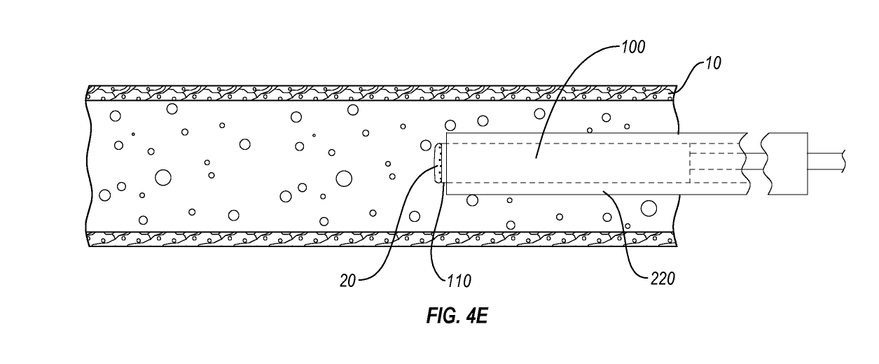

In any event, as described above, the thrombus removal device 100 may engage and secure at least a portion of the thrombus at the capturing portion 110 of the thrombus removal device 100. For example, FIG. 4A illustrates a body lumen 10 and a thrombus 20 located in the body lumen 10. In one example, the thrombus 20 may completely block or occlude the body lumen 10. For instance, the thrombus 20 may prevent or block blood 30 from flowing in the body lumen 10.

The thrombus removal device 100 may engage the thrombus 20 and secure at least a portion thereof, and may remove at least a portion of the thrombus 20. In one instance, the catheter tube 220 together with the thrombus removal device 100 (e.g., in the pre-deployed or collapsed configuration) may be placed within the body lumen 10. Specifically, the thrombus removal device 100 may be located inside the catheter tube 220, and may move together with the catheter tube 220 within the body lumen 10.

For example, the catheter tube 220 may be introduced into the body lumen using the Seldinger technique or the modified Seldinger technique. In the Seldinger technique, a needle may first inserted into the body lumen and a guide wire then follows through the needle. Next, the needle may be removed and a sheath/dilator combination may be advanced over the guide wire. The dilator expands the puncture in the vessel to a size suitable to receive the distal end of an introducer sheath.

After the distal end of the sheath is disposed within the vessel, the dilator and guide wire may be removed, thereby allowing access to the vessel lumen or other body lumen via the inserted introducer sheath. Hence, the catheter tube 220 may be inserted into the body lumen via the introducer sheath. Then, the thrombus removal device 100 may be inserted into the catheter tube 220. Alternatively, the catheter tube 220 together with the thrombus removal device 100 may be introduced together through the introducer sheath.

Subsequently, the catheter tube 220 and the thrombus removal device 100 may be advanced toward and into the thrombus 20, as illustrated in FIG. 4B. For example, the catheter tube 220 and/or the thrombus removal device 100 may include a radiopaque marker, which may aid in guiding the catheter tube 220 as well as the thrombus removal device 100 to a suitable location. Particularly, the catheter tube 220 and the thrombus removal device 100 may be advanced through the thrombus 20, in a manner that the distal end 112 of the thrombus removal device 100 is positioned past the distal side of the thrombus 20. Accordingly, reconfiguring the thrombus removal device 100 and to the deployed configuration may allow the thrombus removal device 100 to engage at least a portion of the thrombus 20. Furthermore, in the deployed configuration, the distal end 112 of the thrombus removal device 100 may be located distally away from the distal side of the thrombus 20, which may increase and/or reestablish fluid flow (e.g., flow of blood 30) through the body lumen 10.

In one embodiment, as described above, the thrombus removal device 100 may be reconfigured into the deployed configuration by moving the thrombus removal device 100 out of the catheter tube 220. For instance, as illustrated in FIG. 4C, the catheter tube 220 may be withdrawn proximally relative to the thrombus removal device 100, while the thrombus removal device 100 may remain substantially stationary relative to the thrombus 20. Accordingly, as the thrombus removal device 100 exits the catheter tube 220, the thrombus removal device 100 may expand into the deployed configuration, thereby engaging the thrombus 20. In particular, the thrombus removal device 100 may engage the thrombus 20 with the capturing portion 110 thereof.

In some instances, at least a portion of the thrombus 20 may pass through the capture cells 140 of the capturing portion 110 and may enter the interior space of the capturing portion 110. Also, a portion of the thrombus 20 may remain positioned between the wall of the body lumen 10 and the capturing portion 110. Alternatively, substantially all of the thrombus 20 may enter the interior space of the capturing portion 110 of the thrombus removal device 100. In any event, however, the capturing portion 110 of the thrombus removal device 100 may engage, connect to, and/or secure the thrombus 20 in a manner that may allow the thrombus removal device 100 to remove the thrombus 20 from the body lumen 10.

In some embodiments, the capturing portion 110 of the thrombus removal device 100 may remain engaged with the thrombus 20 for a predetermined or desired period of time. For instance, the capturing portion 110 may remain engaged with the thrombus 20 for 10-30 minutes, to allow the cells and/or tissue of the thrombus 20 to enter the interior space of the capturing portion 110 and/or to bond or couple to the capturing portion 110 (e.g., within the capture cells 140 of the capturing portion 110). Thereafter, the thrombus removal device 100 together with the thrombus 20 may be reintroduced into the catheter tube 220, as described below in further detail.

In some embodiments, the thrombus removal device 100 also may restore or increase the flow of fluid in the body lumen 10. In particular, the fluid (e.g., blood) located on the proximal side of the thrombus 20 may be channeled through the thrombus removal device 100 and allowed to pass through the thrombus 20. For instance, the fluid may pass through the pass-through cells 180 and may enter the interior space of the pass-through portion 150, which may be in fluid communication with the interior space of the capturing portion 110. As noted above, the distal end 112 of the capturing portion 110 may at least partially protrude past the distal side of the thrombus 20. Accordingly, the fluid entering the thrombus removal device 100 through the pass-through cells 180 and may pass out of the thrombus removal device 100 through the distal end 112 of the capturing portion 110 on the distal side of the thrombus 20.

Also, in some instances, the capturing portion 110 and/or the pass-through portion 150 may be spaced away from the wall of the body lumen 10. In other instances, however, the capturing portion 110 and/or the pass-through portion 150 may abut the wall of the body lumen 10. For example, the capturing portion 110 may expand through the thrombus 20 in a manner that the capturing portion 110 abuts the wall of the body lumen 10. Likewise, the pass-through portion 150 may expand to abut the wall of the body lumen 10. Nevertheless, in the embodiments involving the capturing portion 110 and pass-through portion 150 expanded to abut the wall of the body lumen 10, the fluid may flow through the thrombus removal device 100 and past the thrombus 20.

Embodiments of the present invention also may involve removing the thrombus 20 from the body lumen 10 by reintroducing the thrombus removal device 100 into the catheter tube 220, as illustrated in FIGS. 4D and 4E. More specifically, as the thrombus removal device 100 is reintroduced into the catheter tube 220, the inner dimension (e.g., inside diameter) of the catheter tube 220 may compress or collapse the thrombus removal device 100 into the pre-deployed configuration. Thus, the pass-through portion 150 of the thrombus removal device 100 may be collapsed into the pre-deployed configuration ask the pass-through portion 150 enters the catheter tube 220. Likewise, the capturing portion 110 of the thrombus removal device 100 may be collapsed by the catheter tube 220, as the capturing portion 110 enters the catheter tube 220.

In some embodiments, the catheter tube 220 may be advanced over the thrombus removal device 100, thereby collapsing the pass-through portion 150 and the capturing portion 110. Alternatively, the thrombus removal device 110 may be retracted into the catheter tube 220. Moreover, as the thrombus removal device 110 is retracted into the catheter tube 220, the thrombus removal device 100 may loosen or dislodge and/or move the thrombus 20 toward and into the catheter tube 20.

In any event, the capturing portion 110 may secure the thrombus 20. Thus, as the capturing portion 110 is reintroduced into the catheter tube 220 and is collapsed thereby, the capturing portion 110 may retain at least a portion of the thrombus 20 within the inner space of the capturing portion 110. Accordingly, once the capturing portion 110 is inside the catheter tube 220, at least a portion of the thrombus 20 may be located and secured inside the catheter tube 220 (FIG. 4E).

Subsequently, the catheter tube 220 may be removed from the body lumen 10 together with the capturing portion 110 and with the thrombus 20. Thereafter, the opening created to access the body lumen 10 may be closed to facilitate hemostasis. After removal of the thrombus 20 from the body lumen 10, fluid flow through the body lumen 10 may be increased or restored.

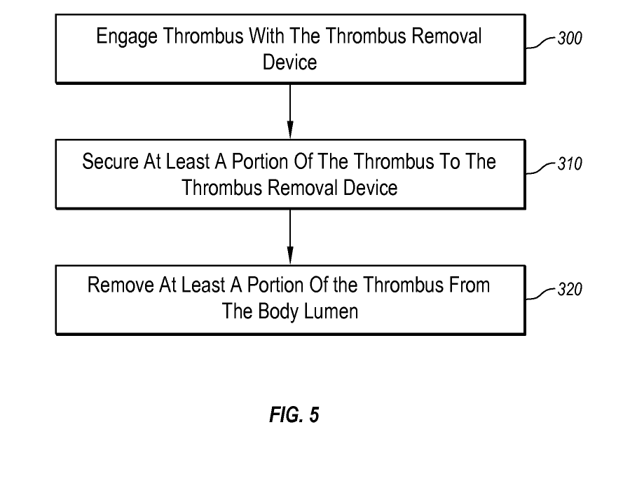

Accordingly, FIGS. 1A-4E and the corresponding text, provide a number of different components, devices, and methods of use thereof for removing thrombus from a body lumen. In addition to the foregoing, embodiments of the present invention may also be described in terms of flowcharts comprising acts and steps in a method for accomplishing a particular result. For example, FIG. 5 illustrates a flowchart of one exemplary method for removing thrombus from a body lumen. The acts of FIG. 5 are described below with reference to the components and diagrams of FIGS. 1A through 4E.

In one embodiment, the method may include an act 300 of engaging the thrombus with the thrombus removal device. For example, the thrombus removal device may be placed within the thrombus in a manner that the capturing portion of the thrombus removal device may engage the thrombus. To place the thrombus removal device within the thrombus, in some instances, a guidewire may be inserted into the body lumen. In some instances, the guidewire may pass through the thrombus. Subsequently, the catheter tube may be inserted over the guidewire and into the body lumen. For instance, the catheter tube may be positioned near the proximal side of the thrombus.

In some embodiments, the thrombus removal device may be fed through the catheter tube. In some instances, the guidewire may be removed before advancement of the thrombus removal device with the catheter tube. Alternatively, the guidewire may remain in the body lumen, and the catheter tube and the thrombus removal device may be advance over the guidewire. Hence, it should be appreciated that, in some embodiments, the guidewire may pass through the thrombus removal device (e.g., through the distal end of the thrombus removal device and out of the connector portion thereof). Moreover, while positioned within the catheter tube the thrombus removal device may be in the pre-deployed or collapsed configuration.

In addition, the thrombus removal device may be placed within the thrombus, and at least a portion of the thrombus removal device may be reconfigured into the deployed or expanded configuration. In some embodiments, the capturing portion of the thrombus removal device may be reconfigured into the deployed configuration, thereby engaging and securing the thrombus. For example, as noted above, the capturing portion of the thrombus removal device may include one or more roughened struts that may define multiple capturing cells. As the capturing portion of the thrombus removal device is reconfigured into the deployed configuration, the roughened struts may press into the thrombus, thereby engaging the thrombus.

By pressing the roughened struts of the capturing portion into the thrombus, at least a portion of the thrombus may be forced into and may enter the capturing cells and/or the inner space of the capturing portion. Moreover, as the roughened struts press into the thrombus, the capturing portion of the thrombus removal device may form a channel in the thrombus. As such, the thrombus removal device may provide a channel or conduit for fluid to flow across the thrombus. Particularly, the fluid may flow through the pass-through cells of the pass-through portion and through the capturing cells and/or the distal end of the capturing portion.

In one embodiment, the thrombus removal device together with the catheter tube may be advanced into the thrombus. For instance, the capturing portion of the thrombus removal device may be positioned within the thrombus. In some instances, the distal end of the capturing portion of the thrombus removal device may be positioned past the distal side of the thrombus. In any event, after positioning the capturing portion of the thrombus removal device inside the thrombus, the capturing portion may be reconfigured from the pre-deployed configuration into the deployed configuration.

For example, as noted above, the thrombus removal device may include memory shape alloys, which may be expanded into the deployed configuration. Thus, in one instance, the catheter tube may be pulled in the proximal direction, while the thrombus removal device is maintained approximately stationary relative to the thrombus. As such, the thrombus removal device may exit the catheter tube and at least a portions thereof (e.g., the capturing portion) may be allowed to expand into the deployed configuration, thereby engaging at least a portion of the thrombus. Additionally or alternatively, the thrombus removal device may be expanded into the deployed configuration using other mechanisms. For instance, an inflatable balloon may be placed within the thrombus removal device and inflated therein, thereby expanding at least a portion of the thrombus removal device (e.g., the capturing portion) into the deployed configuration.

In additional or alternative embodiments, the pass-through portion may be reconfigured into the deployed configuration. As described above, the pass-through portion may be connected to the capturing portion of the thrombus removal device. Moreover, in some embodiments, the capturing portion may be located distally relative to the pass-through portion of the thrombus removal device. Alternatively, however, the capturing portion may be located proximally relative to the pass-through portion of the thrombus removal device. In any event, in at least one embodiment, the entire thrombus removal device (e.g., the pass-through portion and the capturing portion) may be reconfigured from a pre-deployed configuration into a deployed configuration. As mentioned above, reconfiguring the pass-through portion of the thrombus removal device may increase or reestablish fluid flow through the body lumen.

In one embodiment, the method also may include an act 310 of securing at least a portion of the thrombus to the thrombus removal device. For instance, the thrombus may be secured inside the capturing cells and/or inside the inner space of the capturing portion of the thrombus removal device. More specifically, cells and/or tissue comprising the thrombus may bind to and/or may by trapped by the capturing cells and/or inner space of the capturing portion.

In some embodiments, the thrombus removal device may remain stationary or set in place for 10 to 30 minutes after deployment. Stationary time may allow the thrombus to enter or be forced into the inner space of the thrombus removal device and/or to bond to the thrombus removal device. Thus, maintaining the thrombus removal device substantially stationary, with the capturing portion thereof in the deployed configuration inside the thrombus, may facilitate securing the thrombus to the thrombus removal device.

Additionally, reconfiguring the capturing portion of the thrombus removal device from the deployed configuration to the pre-deployed or collapsed configuration may secure at least a portion of the thrombus to and/or inside the capturing portion. For instance, the catheter tube may be advanced distally over the thrombus removal device, thereby collapsing the thrombus removal device into the pre-deployed configuration. Alternatively, the thrombus removal device may be moved proximally, together with at least a portion of the thrombus, and may enter the catheter tube that may reconfigure the thrombus removal device into the pre-deployed configuration. In any event, however, the capturing portion of the thrombus removal device may retain and secure at least a portion of the thrombus, as the thrombus removal device is reconfigured into the pre-deployed configuration. Accordingly, at least a portion of the thrombus may be secured to the thrombus removal device as the thrombus removal device resides within the catheter tube (i.e., at least a portion of the thrombus may be secured inside the catheter tube).

Additionally, the method may include an act 320 of removing at least a portion of the thrombus from the body lumen. As mentioned above, at least a portion of the thrombus may be secured inside the thrombus removal device and/or inside the catheter tube (after collapsing the thrombus removal device therein). Hence, removing the catheter tube together with the thrombus removal device also may remove the portion of the thrombus that is secured by and/or within the thrombus removal device and/or the catheter tube from the body lumen. After removing the catheter tube, the thrombus removal device, and the thrombus (or portion of the thrombus) from the body lumen, the point of access to the body lumen may be closed to establish hemostasis.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. An expandable and collapsible thrombus removal device configured to engage and at least partially remove a thrombus, the thrombus removal device comprising:
a selectively expandable capturing portion including a plurality of capturing cells defined by one or more roughened struts;
a selectively expandable pass-through portion including a plurality of pass-through cells defined by one or more smooth struts, the pass-through portion being connected to the capturing portion, the roughened struts having a higher surface roughness than the smooth struts, and each of the plurality of pass-through cells being larger than each of the plurality of capturing cells;
a connector portion connected to one or more of the capturing portion or the pass-through portion; and
a control wire connected to the connector portion.

2. The thrombus removal device as recited in claim 1, wherein density of capturing cells in the capturing portion is higher than density of pass-through cells in the pass-through portion.

3. The thrombus removal device as recited in claim 1, wherein the one or more smooth struts include a coating.

4. The thrombus removal device as recited in claim 3, wherein the coating includes Poly(vinylidene fluoride-co-hexafluoropropene).

5. The thrombus removal device as recited in claim 1, wherein the one or more roughened struts are uncoated.

6. The thrombus removal device as recited in claim 1, wherein the one or more roughened struts have a positive charge.

7. The thrombus removal device as recited in claim 1, wherein the one or more smooth struts have a negative charge.

8. The thrombus removal device as recited in claim 1, wherein the one or more roughened struts have an Arithmetic Mean Roughness (Rₐ) of greater than 1 μm.

9. The thrombus removal device as recited in claim 1, wherein the one or more smooth struts have an Arithmetic Mean Roughness (Rₐ) of less than 0.5 μm.

10. The thrombus removal device as recited in claim 1, wherein the capturing portion has a tubular shape including an open distal end.

11. The thrombus removal device as recited in claim 10, wherein the pass-through cells of the pass-through portion are in fluid communication with the open distal end of the capturing portion.

12. A method of removing a thrombus from a body lumen, the method comprising;
inserting a capturing portion of a thrombus removal device into the thrombus, while maintaining at least part of a pass-through portion of the thrombus removal device outside of the thrombus, the pass-through portion including a plurality of pass-through cells;
pressing one or more roughened struts of the capturing portion into the thrombus by reconfiguring the capturing portion of the thrombus removal device into a deployed configuration;
receiving at least a portion of the thrombus inside one or more of a plurality of capturing cells of the capturing portion or an inner space of the capturing portion, the plurality of capturing cells being defined by the one or more roughened struts, each of the plurality of the capturing cells being smaller than each of the plurality of pass-through cells; and
securing at least a portion of the thrombus inside one or more of the plurality of capturing cells of the capturing portion or the inner space of the capturing portion.

13. The method of claim 12, wherein inserting the capturing portion of the thrombus removal device into the thrombus includes inserting a catheter tube containing the capturing portion of the thrombus removal device into the thrombus.

14. The method of claim 13, wherein pressing one or more roughened struts of the capturing portion into the thrombus by reconfiguring the capturing portion of the thrombus removal device into a deployed configuration includes withdrawing the catheter tube from the thrombus, while maintaining the capturing portion within the thrombus.

15. The method of claim 14, wherein securing at least a portion of the thrombus inside the inner space of the capturing portion of the thrombus removal device includes reintroducing the capturing portion of the thrombus removal device into the catheter tube.

16. The method of claim 12, further comprising securing at least a portion of the thrombus inside a catheter tube.

17. The method of claim 16, further comprising removing the catheter tube together with at least a portion of the thrombus from the body lumen.

18. The method of claim 12, wherein pressing one or more roughened struts of the capturing portion into the thrombus forms a channel in the thrombus.

19. The method of claim 18, further comprising channeling fluid through the pass-through cells of the pass-through portion, through the opening in the thrombus, and past the thrombus.

20. The method of claim 12, further comprising electrostatically attracting one or more of cells and tissue of the thrombus to the roughened struts.
